(19) United States
(12) Patent Application Publication
(10) Pub. No.: US 2021/0402077 A1
(43) Pub. Date: Dec. 30, 2021

(54) SYSTEM AND METHOD FOR TISSUE SPECIMEN COLLECTION

(71) Applicant: UNITED STATES ENDOSCOPY GROUP, INC., MENTOR, OH (US)

(72) Inventors: Jeffrey B. Cushner, Woodmere, NY (US); Peter M. Kurzyna, Exton, PA (US); Kenneth E. Wolcott, Centerport, NY (US)

(73) Assignee: UNITED STATES ENDOSCOPY GROUP, INC., MENTOR, OH (US)

(21) Appl. No.: 17/368,593

(22) Filed: Jul. 6, 2021

Related U.S. Application Data
(63) Continuation of application No. 14/679,464, filed on Apr. 6, 2015, now abandoned.
Application No. 14/679,464 is a continuation of application No. 11/438,092, filed on May 19, 2006, now abandoned.

(51) Int. Cl. A61M 1/00; A61B 10/00; A61B 10/02; A61B 90/92; A61B 90/96; A61B 90/90; A61B 90/94; A61B 90/98; A61B 1/015; A61B 10/04

(52) U.S. Cl. CPC A61M 1/79; A61B 10/0096; A61B 10/0283; A61B 90/92; A61B 90/96; A61C 17/065; A61B 90/94; A61B 90/98; A61M 1/86; A61B 1/015; A61B 10/04; A61B 90/90

(57) ABSTRACT

A system and method for retaining a tissue specimen obtained via a collection tube is provided. Embodiments provide a system including a collection device removably and serially disposed between a collection tube and a suction tube so as to allow fluid communication therebetween via the collection device. The collection device includes a screen for retaining a tissue specimen drawn into the collection device by and towards the suction tube while allowing any fluid accompanying the tissue to be drawn through the collection device and into the suction tube. The system also includes one or more end caps for sealing the collection device such that the collection device may store and/or transport the retained tissue specimen in a preservation fluid. A system for identifying and organizing collection devices retaining tissue specimens from various anatomical regions is also provided.

### FIELD OF THE INVENTION

The various embodiments of the present invention relate generally to tissue collection devices for retaining a tissue specimen drawn from a patient by a suction device during, for example, an endoscopy procedure.

### BACKGROUND OF THE INVENTION

Endoscopic medical procedures often result in the capture of tissue specimens (such as polyps) recovered from a collection tube in communication with an endoscope during an endoscopy procedure. For example, clinicians often draw tissue specimens through an endoscope and into a collection canister via the application of suction from a suction source positioned proximal to the endoscope. Such tissue specimens are often transported to a pathology laboratory in order to generate a diagnosis based on an analysis of the tissue specimen.

Various conventional tissue specimen traps have been developed for placement between the patient and the collection canister (and a corresponding suction source) in an attempt to separate the tissue specimen from suction effluent that accompanies the tissue specimen from the endoscope. For example, some conventional tissue specimen traps include a “sputum trap” arrangement including a cup or reservoir having a lid defining an inlet (leading to the endoscope) and an outlet (leading to the suction source). Such sputum trap tissue specimen traps may also include one or more baskets or chambers disposed near the inlet for retaining tissue specimens that are drawn into the sputum cup by the suction. However, conventional “sputum cup” tissue specimen traps do not provide direct co-axial fluid communication between the endoscope and the suction source (as both the inlet and outlets of the cup are defined in a “lid” or “cover” of the trap). Thus, suction effluent that accompanies the tissue specimen may not be effectively separated from the tissue specimen retained in the basket or chamber of the trap due to the indirect suction applied to the tissue specimen. For example, the reservoir or “cup” of such conventional traps often retains a considerable volume of potentially contaminating effluent. Furthermore, some conventional sputum cup tissue specimen traps that include multiple tissue specimen chambers (or selectable “baskets”), require the removal of the tissue specimen by an accessory device such as a pair of tweezers.

Some additional conventional tissue specimen traps have been developed to retain a tissue specimen in a position that is substantially co-axial with the suction source and the collection tube. For example, clinicians may insert gauze-like “filters” into a proximal portion of a section of tubing (in communication with the endoscope, for example) using a mandrel or other device. The flexible, porous “filter” is removably engaged between adjacent sections of tubing by the peripheral edges of the “filter” material (which may extend outside the tubing and be retained, for example, by the interaction of serially-engaged sections of endoscope tubing). Such conventional tissue specimen traps, however require a clinician to pull out the tissue specimen out of the gauze-like “filter” retained within the tube or to pull out the entire gauze-like “filter.” Such additional operations may compromise the tissue specimen thereby limiting its value as a diagnostic indicator when examined, for example, by a pathologist. For example, removal tissue specimen from the gauze-like filter could result in the tissue specimen being dropped. In addition, any handling the tissue specimen requires the clinician to immediately transfer the tissue specimen to a secondary container containing a preservative fluid (such as formalin). In addition, the use of flexible fabric or gauze to construct the filter may result in the unwanted retention of effluent in the filter material which may accompany the tissue specimen as it is transferred for downstream transport and/or analysis steps.

The various complications and additional steps required to process tissue specimens retained in conventional tissue specimen traps may thus not only compromise the tissue specimen, but may also result in confusion and/or misidentifying an anatomical location from which the tissue specimen was taken. For example, in multi-chambered “sputum cup” tissue specimen traps, the clinician may be required to quickly retrieve a tissue specimen from one or more chambers or baskets included in the trap. Thus, the clinician may not have adequate time to note the anatomical location from which each tissue specimen may have been drawn. Thus, downstream analysis of the tissue specimen (by an off-site pathologist, for example) may be compromised by mislabeling and/or misidentification of tissue specimens retained in conventional tissue specimen traps.

Thus, there exists a need in the art for a system and method for tissue specimen collection that addresses at least some of the technical issues associated with conventional tissue specimen traps. For example, there exists a need for a single in-line tissue specimen trap that allows for the effective removal of effluent from a tissue specimen retained in the trap and allows a clinician to easily remove the single tissue specimen trap from serial engagement between a collection tube (such as a section of tubing in fluid communication with an endoscope) and a suction source tube and replace with a second single tissue specimen trap. There further exists a need for a tissue specimen trap that may be used as a container for preserving and/or segregating the tissue specimen as it is transported to a pathology laboratory or other facility. Furthermore, there exists a need for a system and method that allows clinicians to easily organize a plurality of tissue specimen traps used in one or more endoscopy procedures based, for example, on an anatomical location from which each tissue specimen is drawn.

### BRIEF SUMMARY OF THE INVENTION

The embodiments of the present invention satisfy the needs listed above and provide other technical advantages as listed below. Embodiments of the present invention may include a tissue specimen collection system comprising a collection device adapted to be removably and serially engaged between a suction tube and a collection tube. The collection device comprises a distal end operably engaged with the collection tube, and a proximal end operably engaged with the suction tube. The collection device also defines a bore extending therethrough, in coaxial relation with the suction tube and the collection tube, for allowing fluid communication between the suction tube and the collection tube. The system further comprises a screen disposed within the bore of the collection device. The screen is configured to retain a tissue specimen drawn through the collection device by and towards the suction tube. The screen defines a plurality of apertures for allowing fluid communication between the collection tube and the suction tube such that any fluid accompanying the tissue specimen is separated and drawn through the screen and into the suction tube, while the tissue specimen is retained by the screen within the collection device.

According to some system embodiments, the collection device is configured to be separable into a first segment (including the distal end, for example) and a second segment (including the proximal end and the screen, for example). Thus, upon separation of the first and second segments, the retained tissue specimen may be accessible for retrieval from the screen by a user of the system. In other system embodiments, the collection device may be configured to serve as a transport container for the tissue specimen retained therein. For example, in some such embodiments, the system may further comprise: a proximal end cap configured to sealingly engage the proximal end of the collection device in a substantially fluid-tight manner when the collection device is removed from serial engagement between the suction tube and the collection tube; and a distal end cap configured to sealingly engage the distal end of the collection device in a substantially fluid-tight manner when the collection device is removed from serial engagement between the suction tube and the collection tube. Thus, the cooperation of the collection device, distal end cap, and proximal end cap may ensure that the tissue specimen is retained within the collection device when the collection device is removed from serial engagement between the suction tube and the collection tube.

Additional system embodiments may further comprise a preservation fluid reservoir configured to operably engage at least one of the proximal end and the distal end of the collection device when the collection device is removed from serial engagement between the suction tube and the collection tube. The preservation fluid reservoir defines an aperture for receiving at least one of the proximal end and the distal end of the collection device and comprises a penetrable membrane configured to substantially seal the aperture so as to contain a preservation fluid within the preservation fluid reservoir. The membrane is configured to be capable of being penetrated by at least one of the proximal end and the distal end of the collection device such that the preservation fluid is released into the bore defined in the collection device to preserve the tissue specimen retained therein.

Some system embodiments further comprise a manifold device configured to removably and serially engage the collection device between the suction tube and the collection tube. The manifold device may comprise at least one valve device operably engaged between the distal end of the collection device and the suction tube, wherein the valve device is configured to selectively allow fluid communication between the collection tube and the suction tube via the collection device. In some such embodiments, the manifold device may be configured to removably and serially engage a plurality of collection devices in parallel relation between the suction tube and the collection tube. Thus, the valve device may be further configured to selectively allow fluid communication between the collection tube and the suction tube via at least one of the plurality of collection devices. The manifold device may also define a bypass bore extending therethrough in coaxial relation with the suction tube and the collection tube. The bypass bore is configured to allow fluid communication between the suction tube and the collection tube. According to such embodiments, the valve device may be further configured to selectively allow fluid communication between the collection tube and the suction tube via the bypass bore so as to bypass the collection device (or a plurality of collection devices in parallel relation between the suction tube and the collection tube).

According to some system embodiments of the present invention, the collection device may comprise at least one indicia corresponding to an anatomical region from which the tissue specimen is drawn. Thus, such embodiments may allow a user of the system to identify the anatomical region from which the tissue specimen was drawn during a medical procedure. In various embodiments, the at least one indicia may include, but is not limited to: an alphanumeric indicia (affixed to the collection device, for example); a color; a bar code; a radio-frequency identification (RFID) device; and combinations of such indicia. In order to provide improved organization and/or identification of the collection device (and tissue specimens retained therein) after removal of the collection device from serial engagement between the suction tube and the collection tube, some system embodiments may further comprise an organizer device configured to removably and serially engage the collection device between the suction tube and the collection tube. The organizer device also defines a plurality of apertures for receiving the collection device when the collection device (retaining the tissue specimen, for example) is removed from serial engagement between the suction tube and the collection tube. The plurality of apertures include one or more anatomical indicia corresponding thereto for indicating an anatomical region from which the tissue specimen is drawn such that the anatomical region is identifiable based at least in part on the anatomical indicia.

Various other embodiments of the present invention may also provide methods for collecting a tissue specimen. In one embodiment, the method comprises providing a collection device adapted to be removably and serially engaged between a suction tube and a collection tube, wherein the collection device defines a bore extending therethrough in coaxial relation with the suction tube and the collection tube. As described herein, the bore is configured to allow fluid communication between the suction tube and the collection tube. The provided collection device also includes a screen disposed within the bore, defining a plurality of apertures for allowing fluid communication between the collection tube and the suction tube. The method also comprises: operably engaging a distal end of the collection device with the collection tube; operably engaging a proximal end of the collection device with the suction tube; drawing a tissue specimen through the collection tube by and towards the suction tube; and retaining the tissue specimen on the screen such that such that any fluid (such as effluent) accompanying the tissue specimen is separated and drawn through the screen and into the suction tube.

Other method embodiments further comprise separating the collection device into a first segment including the distal end and a second segment including the proximal end and the screen, retrieving the retained tissue specimen from the screen, and submerging the retrieved tissue specimen in a preservation fluid. Other method embodiments may include steps for utilizing the provided collection device as a tissue specimen storage and/or transport container. For example, the method may further comprise operably engaging a proximal end cap with the proximal end of the collection device in a substantially fluid-tight manner when the collection device is removed from serial engagement between the suction tube and the collection tube. The method may also comprise operably engaging a distal end cap with the distal end of the collection device in a substantially fluid-tight manner when the collection device is removed from serial engagement between the suction tube and the collection tube, and transporting the retained tissue specimen to a laboratory within the collection device (which, as described herein, is substantially closed by the proximal and distal end caps operably engaged therewith). In order to preserve the retained tissue specimen as it is stored and/or transported within the collection device, various method embodiments of the present invention may also comprise filling the collection device with a preservation fluid (such as formalin, for example) so as to preserve the retained tissue specimen within the collection device.

Thus, the various embodiments of the present invention provide many advantages that may include, but are not limited to: allowing a clinician to quickly change tissue specimen collection traps without having to immediately transfer the tissue specimen to a separate preservation fluid container; providing a self-contained, enclosed tissue specimen collection device which may be stored prior to transport to a pathology laboratory such that multiple tissue specimens collected during a single endoscopy procedure may be stored and transferred at one time; providing a self-contained, small and portable tissue specimen collection device that allows for an improved separation of effluent from the retained tissue specimen via the application of a suction force that is substantially co-axial with the collection device; and providing a system and method for organizing and identifying multiple tissue specimen collection devices based at least in part on the anatomical location from which the tissue specimen is drawn.

These advantages, and others that will be evident to those skilled in the art, are provided in the system and method for tissue specimen collection of the present invention.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 shows several views of a system according to one embodiment of the present invention comprising a collection device operably engaged with a collection tube;

FIG. 2A shows a system according to one embodiment of the present invention comprising a collection device operably engaged with distal and proximal end caps;

FIG. 2B shows a system according to one embodiment of the present invention comprising a collection device and a collection tube wherein the ends of the collection tube are operably engaged with the distal and proximal ends of the collection tube;

FIG. 3 shows a system according to one embodiment of the present invention comprising a collection device operably engaged with a preservation fluid reservoir;

FIG. 4 shows a system according to one embodiment of the present invention comprising a pair of collection devices operably engaged with a manifold device;

FIG. 5 shows a system according to one embodiment of the present invention comprising a collection device operably engaged with an organizer device defining apertures for receiving collection devices having tissue specimens retained therein;

FIG. 6 shows a system according to one embodiment of the present invention comprising a collection device operably engaged with an alternate organizer device defining apertures for receiving collection devices having tissue specimens retained therein;

FIG. 7 shows a system according to one embodiment of the present invention comprising a collection device operably engaged with a manifold device defining a reservoir;

FIG. 8 shows a system according to one embodiment of the present invention comprising a collection device operably engaged with a manifold device defining a reservoir wherein the collection device is oriented such that a gravity force acts to drain a fluid from the collection device;

FIG. 9 shows a system according to one embodiment of the present invention comprising a collection device operably engaged with an alternative manifold device defining a reservoir wherein the collection device is oriented such that a gravity force acts to drain a fluid from the collection device;

FIG. 10 shows a system according to one embodiment of the present invention comprising a collection device operably engaged with a manifold device via a cartridge device that is slidably engaged with a cartridge aperture defined in the manifold device; and

FIG. 11 shows a cartridge device for receiving the collection device that is adapted to be slidably engaged with a cartridge aperture defined in the manifold device, according to one embodiment of the present invention.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

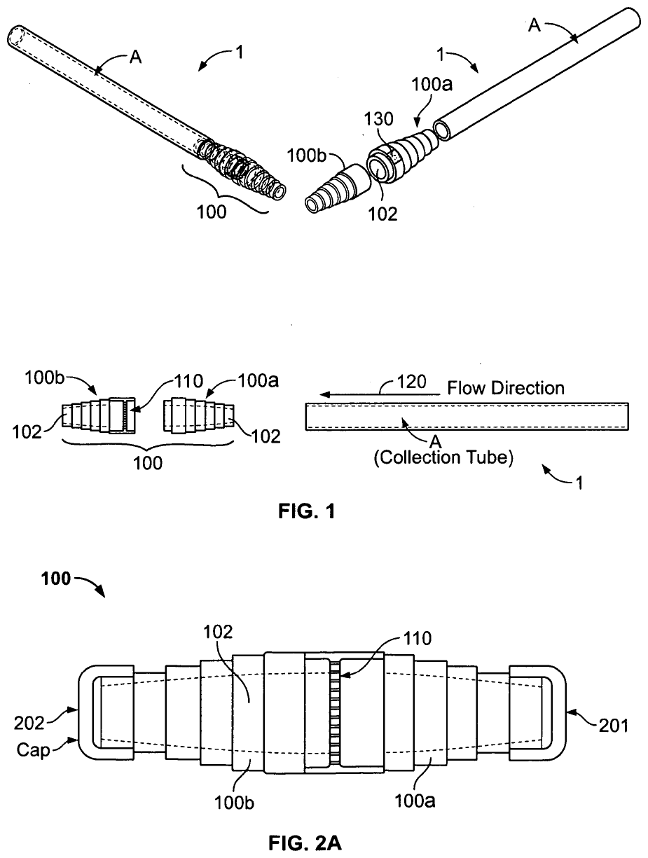

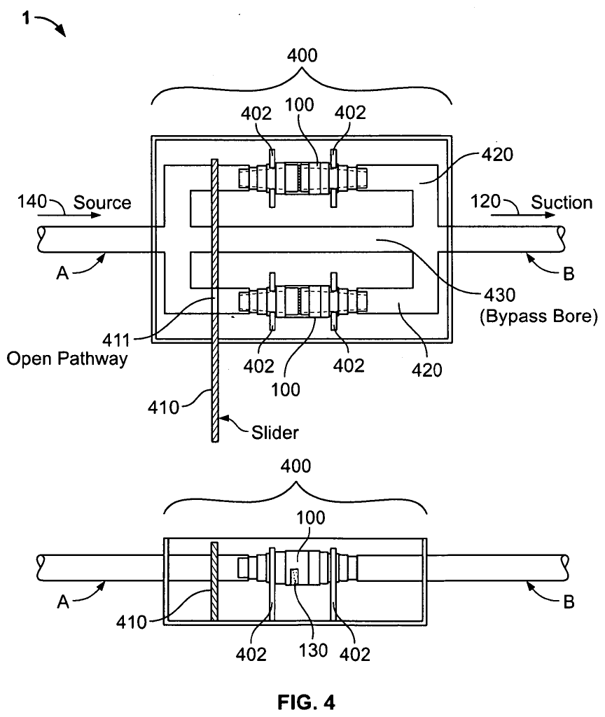

As shown generally in FIG. 1, one embodiment of the present invention provides a tissue specimen collection system 1 comprising a collection device 100 adapted to be removably and serially engaged between a suction tube B (see FIG. 4) and a collection tube A. For example, in some embodiments, the collection device 100 may be adapted to be removably and serially engaged between an endoscope collection tube A and a suction tube B such that a flow direction 120 is established through the collection device 100 (and/or a bore 102 defined therein) via a suction force imparted by a suction source in fluid communication with the collection device 100 via the suction tube B (see FIG. 4).

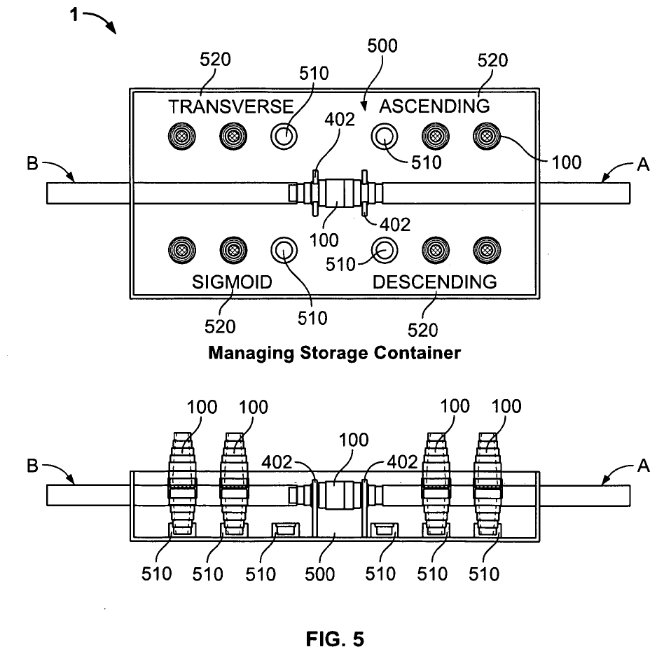

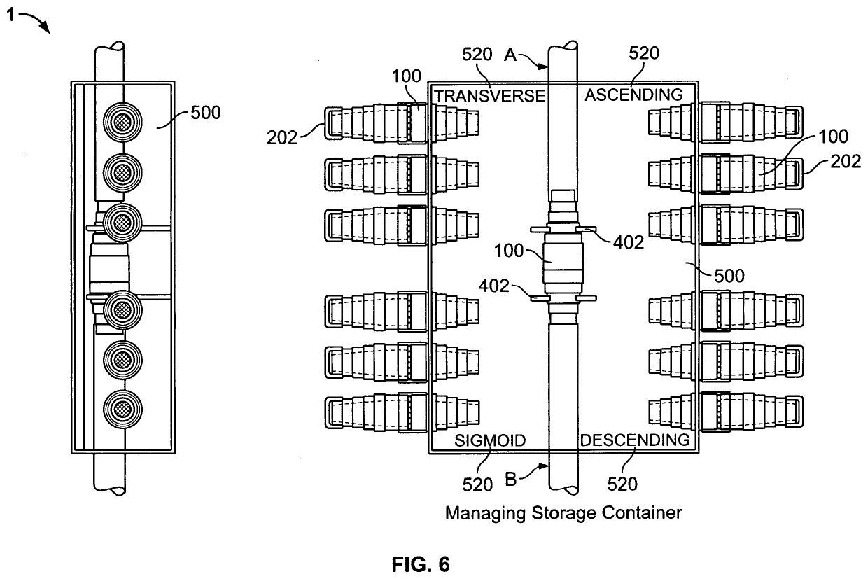

The collection device 100 comprises a distal end operably engaged with the collection tube A and a proximal end operably engaged with the suction tube B (see FIG. 4, for example). As shown generally in FIG. 1, the distal and proximal ends of the collection tubes may define one or more steps having varying outer diameters configured to establish an interference fit within collection tubes A and/or suction tubes B having a variety of inner diameters. In other embodiments, the distal and proximal ends of the collection tubes may define barbs, threaded surfaces, and/or other quick-connection devices configured to removably and serially engage the collection device 100 between collection tubes A and/or suction tubes B having a variety of inner diameters. Thus, the collection device 100 may be easily removable from serial engagement between the collection tube A and the suction tube B by a user of the system 1. When a tissue specimen is retained within the collection device 100 (as described further herein), a user may quickly disengage a collection device 100 retaining a tissue specimen and replace the collection device 100 with an empty collection device 100 that may be relatively easily serially engaged between the collection tube A and the suction tube B. Thus, according to various system 1 embodiments of the present invention, a single tissue specimen may be captured and retained in a corresponding single collection device 100 that may be organized based upon, for example, an anatomical region from which the tissue specimen is drawn (as discussed further herein with respect to FIGS. 5 and 6 (depicting a system 1 embodiment further comprising an organizer device 500 for organizing filled collection devices 100 after each collection device 100 is removed from serial engagement between the collection tube A and the suction tube B). The collection device 100 may also be removed from serial engagement between the collection tube A and the suction tube B and placed directly into a specimen jar or other container containing a preservation fluid (such as formalin, for example) such that the tissue specimen may be contained and submerged for transport.

The collection device 100 defines a bore 102 extending therethrough in coaxial relation with the suction tube B and the collection tube A. The bore 102 defined by the collection device 100 is configured to allow fluid communication between the suction tube B and the collection tube A. In some embodiments, the bore 102 may be configured to have an outer dimension (i.e. diameter) substantially equivalent to a diameter of a channel defined in the suction tube B and/or the collection tube A. In order to retain a tissue specimen, the system 1 further comprises a screen 110 disposed within the bore 102 of the collection device 100. The screen 110 is configured to retain a tissue specimen drawn through the collection device 100 by and towards the suction tube B (i.e. with the flow direction 120 established, for example, by a suction source). The screen 110 defines a plurality of apertures for allowing fluid communication between the collection tube A and the suction tube B (see FIG. 4) such that any fluid accompanying the tissue specimen is separated and drawn through the screen 100 and into the suction tube B while the tissue specimen is retained by the screen 110 within the collection device 100. Because, as shown generally in FIG. 1, the collection device 100 defines a bore 102 that is substantially co-axial with flow channels defined by the collection tube A and the suction tube B, the suction force (establishing a predominant flow direction 120) may effectively draw effluent and/or other fluids accompanying the tissue specimen through the screen 110 (as substantially all of the cross-sectional area of the screen 110 is exposed to the suction force in the flow direction 120) while the screen 110 retains the tissue specimen (such as a polyp retrieved during a colonoscopy or other endoscopic procedure). According to various embodiments of the system 1, the apertures defined by the screen 110 may be sized to retain tissue specimens of a selected size while allowing fluids (such as effluents containing small particulates) to pass through the screen 110 and into a collection canister or other waste receptacle downstream of the collection device 100.

As shown generally in FIG. 1, the collection device 100 (and the bore 102 defined therein), may be formed with a substantially circular cross-section. However, in other embodiments, the collection device 100 (and the bore 102 defined therein), may be formed with various cross-sectional shapes that may include, but are not limited to: rectangular, oval, triangular, and combinations thereof. Furthermore, the collection device 100 (and components thereof, such as the screen 110, for example) may be formed of various materials that may include, but are not limited to: molded polymer; extruded polymer; metal; metallic alloy; and combinations thereof. For example, in one embodiment, the collection device 100 (and the screen 110 disposed therein) may be formed of a substantially transparent, regulatory-compliant polymer (such as medical-grade polycarbonate) such that a clinician may observe the passage of effluent through the bore 102 of the collection device and the retention of a tissue specimen (such as a polyp) by the screen 110 disposed therein. The screen 110 may be integrated with the collection device 100 structure (i.e. integrally formed and/or molded as part of the collection device 100). In other embodiments, the screen 110 may be formed separately from the collection device 100 and inserted into the bore 102 of the collection device 100 so as to be capable of retaining tissue specimens that are drawn through the collection device 100 (by the suction tube B, for example). The screen 110 may thus be formed of the same materials as the structure of the collection device 100. In other embodiments, the screen 110 may comprise various substantially porous metallic and/or polymer screen materials defining apertures for allowing fluid communication between the sides of the screen 110.

As described herein, the collection device 100 may also comprise a substantially-transparent tinted polymer such that the polymer is tinted with a color that may be indicative of an anatomical location from which a particular tissue specimen is drawn. Thus, collection devices 100 may be color-coded to match the apertures 510 (corresponding to anatomical indicia 520) defined in an organizer device 500 (as shown, for example, in FIGS. 5 and 6). Thus, the color-coded collection devices 100 may allow for easy identification and placement in the organizer device 500. Color coding of the collection device 100 may also help eliminate confusion and accidental “mix-ups” of the retained tissue specimen with an incorrect anatomical location. For example, a clinician may serially engage a red-tinted collection device 100 between the collection tube A and the suction tube B for endoscopy procedures in the sigmoid colon of a patient. Other colors may be used to indicate that a particular collection device 100 contains a tissue specimen drawn from a variety of anatomical locations that may include, but are not limited to: ascending colon; descending colon; transverse colon; and other anatomical regions that may be investigated as part of a colonoscopy or other endoscopy procedure utilizing a collection tube A and corresponding suction tube B.

Furthermore, the collection device 100 may comprise at least one indicia (such as a marking and/or label operably engaged with a surface of the collection device 100) corresponding to an anatomical region from which the tissue specimen is drawn such that a user of the tissue specimen collection system 1 may identify the anatomical region from which the tissue specimen was drawn during a medical procedure (such as an endoscopy procedure). According to various embodiments, the at least one indicia of the collection device may include, but is not limited to: an alphanumeric indicia; a color (such as a tint added to a polymer used to form the collection device 100); a bar code; a radio-frequency identification (RFID) device; and combinations of such indicia. In embodiments wherein the indicia comprises an RFID device integrated with the collection device 100 a user of the system 1 may utilize an RFID transmitter/receiver device (in communication with a computer device, for example) for writing data to the RFID device that may include, but is not limited to: patient identifying information; procedure identifying information (procedure name, time, date, etc.); anatomical region; and/or combinations of such data.

Furthermore, in other embodiments, the collection device 100 may comprise other markings operably engaged therewith. For example, a flow direction indicator (such as an arrow pointing in the intended flow direction 120) may be printed on a surface of the collection device such that a user of the system 1 may properly serially engage the collection device 100 between the collection tube A and the suction tube B such that the screen 110 disposed in the bore 102 of the collection device 100 may retain the tissue specimen.

In some system 1 embodiments of the present invention, as shown generally in FIG. 1, the collection device 100 may be configured to be separable into a first segment 100a (including the distal end of the collection device 100), and a second segment 100b (including the proximal end of the collection device 100 and the screen 110). Thus, according to such embodiments, the screen 110 may readily accessible to a clinician or technician such that the tissue specimen (retained by the screen 110 disposed within the second segment 100b) is accessible for retrieval when the first segment 100a is separated from the second segment 100b. In some embodiments, the separated second segment 100b (and the tissue specimen retained on the screen 110 included therein) may also be placed directly into a specimen jar or other container containing a preservation fluid (such as formalin, for example) such that the tissue specimen may be contained and submerged for transport. In other embodiments, once the first segment 100a is separated from the second segment 100b, a clinician may retrieve the tissue specimen directly from the screen 110 (using a retrieval tool including, but not limited to: forceps; tweezers; and/or a pipette) and placed directly into a specimen jar or other container containing a preservation fluid such that the tissue specimen may be contained and submerged for transport to a pathology laboratory.

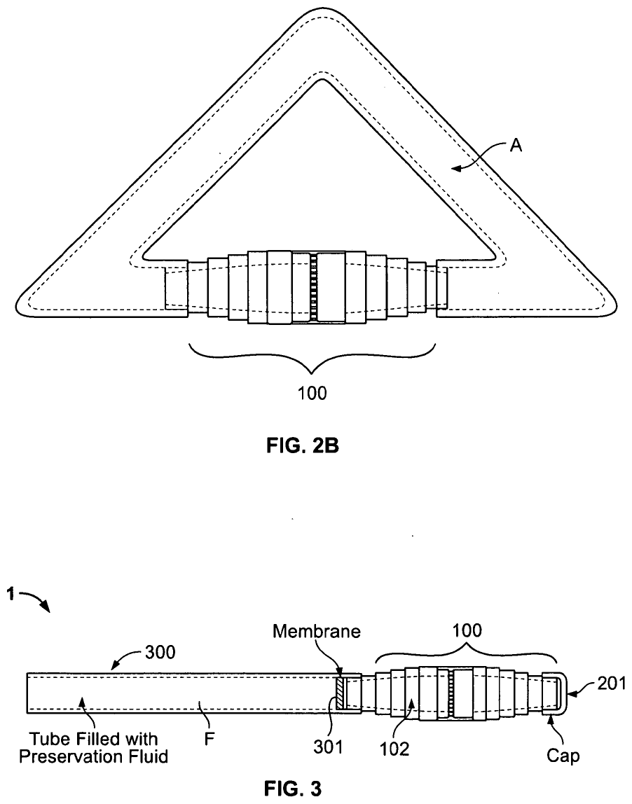

According to some embodiments, as shown generally in FIG. 2A, the tissue specimen collection system 1 may further comprise a distal end cap 201 configured to sealingly engage the distal end of the collection device 100 in a substantially fluid-tight manner such that the tissue specimen is retained within the collection device between the screen 110 and the distal end cap 201 when the collection device 100 is removed from serial engagement between the suction tube B and the collection tube A. Furthermore, as shown in FIG. 2A, the system 1 may also further comprise a proximal end cap 202 configured to sealingly engage the proximal end of the collection device 100 in a substantially fluid-tight manner such that a preservation fluid (such as formalin, for example) may be retained within the collection device 100 between the distal end cap 201 and the proximal end cap 202 to preserve the tissue specimen when the collection device 100 is removed from serial engagement between the suction tube B and the collection tube A. For example, after the collection device 100 is removed from serial engagement between the suction tube B and the collection tube A, the distal end cap 201 may be operably engaged with the distal end of the collection device 100 to ensure that the tissue specimen retained therein does not emerge from the distal end of the collection device. A preservation fluid (such as formalin, for example) may then be introduced into the bore 102 of the collection device 100 via the open proximal end of the collection device 100 in order to submerge the retained tissue specimen in the preservation fluid. Finally, in order to substantially seal the collection device 100 to serve as a container for transporting and/or storing the retained tissue specimen, the proximal end cap 202 may be operably engaged with the proximal end of the collection device 100 such that the preservation fluid is retained within the collection device 100 between the distal end cap 201 and the proximal end cap 202 during subsequent storage and/or transport. As shown generally in FIG. 2B, wherein the collection tube A and/or suction tube B comprises a substantially flexible material (such as silicone tubing or other flexible polymer tubing, for example), a length of collection tube A (and/or a length of suction tube B) may be cut and bent such that one end of the tube A, B is operably engaged with a distal end of the collection device 100 and the other end of the tube A, B is operably engaged with the proximal end of the collection device 100. Thus the tube A, B may form a substantially continuous and closed fluid circuit with the bore 102 defined in the collection device 100 such that the retained tissue specimen (and a volume of preservation fluid) may be retained within the collection device 100 (and within the section of tube A, B).

FIG. 3 shows an additional system 1 embodiment of the present invention wherein the system 1 further comprises a preservation fluid reservoir 300 configured to operably engage at least one of the proximal end and the distal end of the collection device 100 when the collection device is removed from serial engagement between the suction tube B and the collection tube A. As shown in FIG. 3, the preservation fluid reservoir 300 defines an aperture for receiving at least one of the proximal end and the distal end of the collection device 100. Furthermore, the preservation fluid reservoir 300 comprises a penetrable membrane 301 configured to substantially seal the aperture so as to contain a preservation fluid F (such as formalin, for example) therein. The penetrable membrane 301 is configured to be capable of being penetrated by at least one of the proximal end and the distal end of the collection device 100 such that the preservation fluid F is released into the bore 102 defined in the collection device 100 to preserve the tissue specimen retained therein. Thus, as shown in FIG. 3, the preservation fluid reservoir 300 may cooperate with a distal end cap 201 to retain the preservation fluid F within a channel of the preservation fluid reservoir and/or within the bore 102 of the collection device 100 so as to submerge the retained tissue specimen in the preservation fluid F for storage and/or transportation of the collection device 100/preservation fluid reservoir 300 subassembly.

The aperture defined in the preservation fluid reservoir 300 may be configured to receive and secure at least one end of the collection device 100 in a fluid-tight interference fit. As described herein with respect to the serial engagement of the collection device 100 between the collection and suction tubes A, B, the ends of the collection device 100 may comprise a stepped plurality of diameters configured to be capable of being received within a variety of aperture diameters that may be defined by one or more different preservation fluid reservoirs 300. Furthermore, as shown in FIG. 3, the preservation reservoir 300 may comprise a tube defining a channel having a substantially circular cross-section. The preservation reservoir 300, however, may comprise a variety of different containers defining an aperture for receiving an end of the collection device 100. Such container types may include, but are not limited to: vials; bottles; flexible polymer bags; flasks; and combinations of such container types.

FIG. 4 shows an alternate embodiment of the system 1 of the present invention further comprising a manifold device 400 configured to removably and serially engage the collection device 100 between the suction tube B and the collection tube A. For example, the manifold device 400 may comprise one or more resilient structures 402 (such as elastic C-clips and/or blocks of elastomeric polymer defining apertures for receiving and/or sealing one or more ends of the collection device 100) for removably engaging the collection device 100 with a body of the manifold device 400. As shown in FIG. 4, the manifold device 400 may comprise at least one valve device 410 operably engaged between the distal end of the collection device 100 and the suction tube B and configured to selectively allow fluid communication between the collection tube A and the suction tube B via the collection device 100 (and/or a bore 102 defined therein). For example, the valve device 410 may comprise a slidable barrier (configured to be slidable in a direction substantially perpendicular to the flow direction 120, for example) defining a valve aperture 411 sized to substantially approximate the diameter of the bore 102 of the collection device 102. Thus, when the valve device 410 is positioned to allow fluid communication between the collection tube A and the suction tube B via the collection device 100 (i.e. by positioning the valve aperture 411 in line with the bore 102 of the collection device 100) a tissue specimen (and/or fluid accompanying such a specimen) may be drawn through the collection device 100 via the suction force created by the suction tube 100 (and/or a suction source in fluid communication therewith).

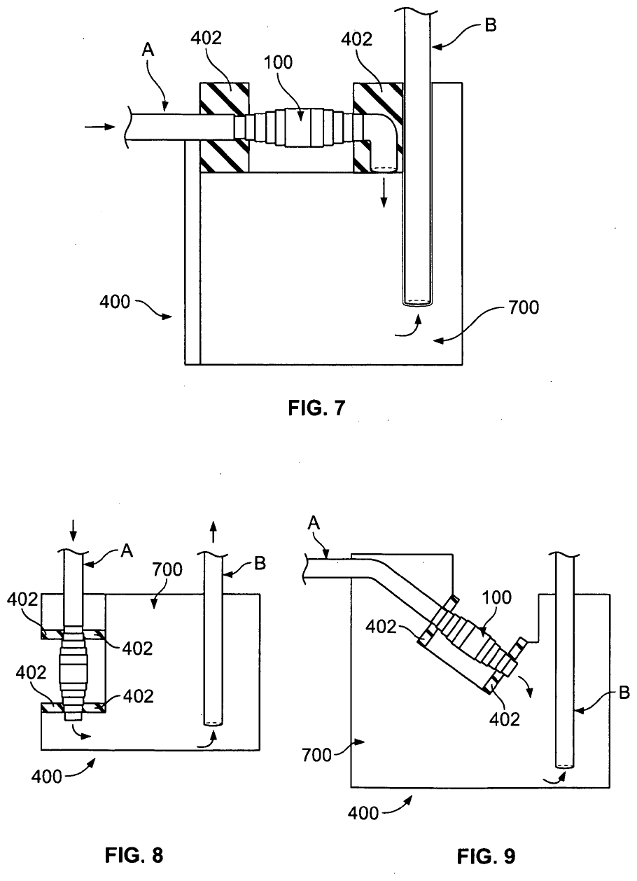

In some embodiments, as shown generally in FIGS. 7-11, the manifold device 400 defines a reservoir 700 in fluid communication between the proximal end of the collection device 100 and the suction tube B. For example, as shown in FIGS. 7-9, the manifold device 400 may define a suction aperture for receiving the suction tube B such that the suction tube extends into the reservoir 700. As described further herein, the proximal end of the collection device 100 may be operably engaged with a substantially resilient structure 402 (such as an elastic polymer material defining an aperture configured to create a fluid tight seal around the proximal end of the collection device 100) such that substantially leak-free fluid communication may be established between a proximal end of the collection device 100 and an entrance aperture defined in the manifold device 400. The reservoir 700 may be configured to receive any fluid accompanying the tissue specimen such that the fluid may be drawn into the suction tube B by the application of a suction force. Thus, as a tissue specimen is drawn from the collection tube A and into the collection device 100 disposed at an entrance to the manifold device 400, the tissue specimen is retained on the screen 110 within the bore 102 of the collection device 100 and any fluid accompanying the tissue sample is drawn into the reservoir 700 and subsequently through the suction tube B by the application of a suction force via the suction tube B.

As shown in FIGS. 7-9, the manifold device 400 may comprise at least one substantially resilient structure 402 configured to removably and serially engage the collection device 100 between the suction tube B and the collection tube A. For example, the resilient structure 402 may include, but is not limited to: a substantially-resilient and/or elastic polymer block defining an aperture being configured to receive at least one of the proximal and distal ends of the collection device 100 (such that the polymer block 402 (see FIG. 8, for example) may establish fluid communication between the collection tube A and the collection device 100; a substantially-resilient C-clip being configured to receive at least one of the proximal and distal ends of the collection device 100 (see generally, the C-clip resilient structure 402 shown operably engaged with the cartridge device 800 in FIG. 11); and combinations of such substantially resilient structures 402. As shown in FIGS. 8 and 9, the manifold device 400 (and the substantially resilient structures 402 included in some embodiments thereof) may be configured to orient the collection device 100 substantially vertically (see generally, FIG. 8) and/or at an acute angle (see generally, FIG. 9) such that a gravity force may aid the drainage of fluid from the collection device 100 (in addition to the exertion of a suction force via the suction tube B for example).

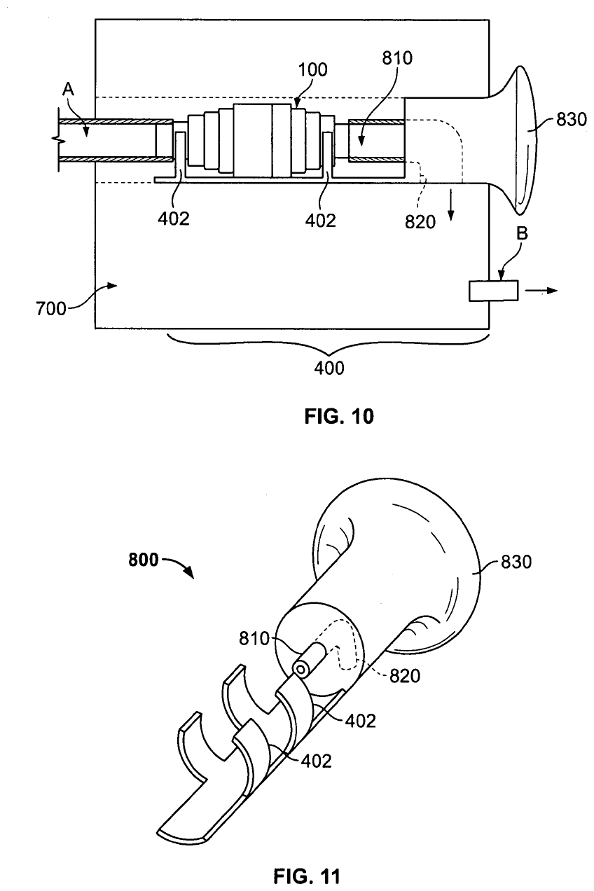

Furthermore, and as shown generally in FIGS. 10 and 11, the system 1 may further comprise a cartridge device 800 configured to removably and serially engage the collection device 100 between the reservoir 700 and the collection tube A. The cartridge device 800 may be configured to be slidably disposed in a cartridge chamber defined in a surface of the manifold device 400 such that the cartridge device 800 is selectively movable between a first position (see FIG. 10, for example) and a second position relative to the manifold device 400. For example, as shown in FIG. 10, the distal end of the collection device 100 may be in direct fluid communication with the collection tube A (via, for example, an aperture defined in a resilient structure 402 (as shown in FIG. 7)) when the cartridge device is in the first position. Furthermore, the cartridge device 800 may comprise a handle 830 defining an exit channel 810 that may be in fluid communication with a proximal end of the collection device 100. The exit channel 810, as shown in FIG. 10, may be configured to direct any fluid separated from the retained tissue sample into the reservoir 700 defined by the manifold device 400 via an exit aperture 820 defined in a lower portion of the cartridge device 800. Furthermore, as shown in FIG. 11, the cartridge device 800 may be configured to be slidably removed from the manifold device 400 (and/or slidably extended from the manifold device 400) such that a user of the system may have access to the substantially resilient structures 402 (such as one or more C-clips) for removing and/or replacing a collection device 100 from the cartridge device 800 during and/or after a medical procedure.

In some system 1 embodiments, the manifold device may be configured to removably and serially engage a plurality of collection devices 100 (such as a pair of collection devices 100, as shown generally in FIG. 4) in parallel relation between the suction tube B and the collection tube A. Furthermore, the valve device 410 may be further configured to selectively allow fluid communication between the collection tube A and the suction tube B via at least one of the plurality of collection devices. Thus, by actuating the valve device 410, a user (such as a clinician performing and/or assisting in an endoscopy procedure) may select one or more of the collection devices 100 serially engaged (in the manifold device 400) in parallel relation between the suction tube B and the collection tube A. As shown in FIG. 4, the manifold device 400 may also define a bypass bore 430 extending therethrough in coaxial relation with the suction tube B and the collection tube A. The bypass bore 430 is configured to allow fluid communication between the suction tube B and the collection tube A when, for example, the valve aperture 411 of the valve device 410 is aligned with the bypass bore 430. As shown in FIG. 4, the valve device 410 may be further configured to selectively allow fluid communication between the collection tube A and the suction tube B via the bypass bore 430 so as to bypass one or more of the plurality of collection devices 100 disposed in parallel within the manifold device 400.

Thus, according to some system 1 embodiments, as shown generally in FIG. 4, manifold device 400 may allow a user to pre-place multiple collection devices 100 in a single manifold device 400. Furthermore, as described herein, the user may actuate the valve device 410 (such as a manual sliding barrier) to switch between individual collection devices 100 that may be serially engaged (within the manifold device 400) between the collection and suction tubes A, B without having to physically replace individual collection devices 100. As shown in FIG. 4, the manifold device 400 may be generally rectangular and include a slidable valve device 410 defining an aperture 411 that may be actuated linearly so as to be aligned with one or more of the bores 102 defined in the collection devices 100. In other embodiments, the manifold device 400 may comprise a substantially cylindrical chamber configured to serially engage a plurality of collection devices 100 in parallel relation between the collection and suction tubes A, B. According to such embodiments, the valve device 410 may comprise a rotatable disc or other barrier defining a single aperture 411 that may be selectively aligned with one or more of the plurality of collection devices 100 arranged in a substantially circular pattern within the cylindrical manifold device 400. Furthermore, while the valve devices 410 shown in FIG. 4 is manually operated, it should be understood that the valve device 410 may also comprise one or more pneumatic, electromechanical, and/or solenoid-operated valve devices for selectively allowing fluid communication between the collection and suction tubes A, B via one or more individual collection devices 100 disposed within the manifold device 400.

FIGS. 5 and 6 show an alternative embodiment of the system 1 of the present invention further comprising an organizer device 500 configured to removably and serially engage the collection device 100 between the suction tube B and the collection tube A. As shown in FIGS. 5 and 6, the organizer device 500 may comprise a plurality of resilient structures 402 (such as substantially elastic polymeric and/or metallic “C” clips) for operably engaging the collection device 100 with the organizer device 500 and for serially engaging the collection device 100 between the suction tube B and the collection tube A. The organizer device 500 defines a plurality of apertures 510 therein for receiving the collection device 100 when the collection device 100 (retaining the tissue specimen) is removed from serial engagement between the suction tube B and the collection tube A. As shown in FIGS. 5 and 6, the plurality of apertures 510 may include one or more anatomical indicia 520 corresponding thereto for indicating an anatomical region from which the tissue specimen is drawn such that the anatomical region is identifiable based at least in part on the anatomical indicia 520. For example, the embodiment of the organizer device 500 shown generally in FIG. 5 includes twelve apertures 510 divided into four different anatomical regions (each having a corresponding anatomical indicia 520). Thus, referring to FIG. 5, a collection device 100 containing a tissue specimen drawn from a patient's sigmoid colon may be placed in an aperture 510 in the region of the organizer device 500 corresponding to the “sigmoid” anatomical indicia 520 (i.e. the lower left portion of the organizer device 500 shown in FIG. 5). Thus, the organizer device 500 may allow a user to quickly and easily associate retained tissue specimens with the anatomical location from which the specimen was removed. As described herein, the collection devices 100 may be further keyed by color or other indicia 130 to ensure that the collection device 100 is properly placed within the organizer device 500.

Various embodiments of the present invention may also provide methods for collecting a tissue specimen in a collection device disposed between a collection tube A (such as a section of tubing in fluid communication with an endoscope or other medical instrument, for example) and a suction source (configured to draw a vacuum and/or create a suction force within a suction tube B. According to some embodiments, the method first comprise providing a collection device 100 such as that described herein with respect to the system 1 embodiments of the present invention (shown, for example, in FIGS. 1-6). The provided collection device 100 is adapted to be removably and serially engaged between a suction tube B and a collection tube A. The provided collection device 100 defines a bore 102 extending therethrough in coaxial relation with the suction and collection tubes B, A. The bore 102 is configured to allow fluid communication between the suction and collection tubes B, A. Furthermore, the provided collection device 100 comprises a screen 110 disposed within the bore 102 defining a plurality of apertures for allowing fluid communication between the collection tube A and the suction tube B.

The method further comprises operably engaging a distal end of the collection device 100 with the collection tube A and operably engaging a proximal end of the collection device 100 with the suction tube B. As shown generally in FIG. 1, the operably engaging steps may be performed by inserting the ends of the collection device 100 into channels defined in the respective ends of the collection and suction tubes A, B. The method further comprises drawing a tissue specimen through the collection tube by and towards the suction tube B (for example, by applying a suction force within a channel defined by the suction tube B using a suction source (not shown)). The method further comprises retaining the tissue specimen on the screen 110 such that any fluid accompanying the tissue specimen is separated, and drawn through the screen 110 and into the suction tube B.

According to some additional method embodiments, the method further comprises separating the collection device 100 into a first segment 100a including the distal end and a second segment 100b including the proximal end and the screen 110. Such method embodiments further comprise: retrieving the retained tissue specimen from the screen 110 (using, for example, a tool such as a pair of tweezers or forceps); and submerging the retrieved tissue specimen in a preservation fluid (such as formalin, for example). The preservation fluid may be contained, for example, in a specimen jar or other container configured to retain the preservation fluid and the retained tissue specimen for storage and/or transport to a pathology laboratory.

Some method embodiments of the present invention may alternatively comprise utilizing the provided collection device 100 as a container for storing and/or transporting the retained tissue specimen to a pathology laboratory for inspection and/or analysis. For example, such embodiments may comprise operably engaging a distal end cap 201 with the distal end of the collection device 100 in a substantially fluid-tight manner such that the tissue specimen is retained within the collection device 100 (between the screen 110 and the distal end cap 201) when the collection device 100 is removed from serial engagement between the suction tube B and the collection tube A. Furthermore, in order to ensure that the collection device 100 is capable of retaining preservation fluid (such as formalin, for example) therein, some method embodiments may further comprise operably engaging a proximal end cap 202 with the proximal end of the collection device 100 in a substantially fluid-tight manner such that the tissue specimen is retained within the collection device 100 between the distal end cap 201 and the proximal end cap 202 when the collection device 100 is removed from serial engagement between the suction tube B and the collection tube A (see, for example, FIG. 2A). The method may also comprise transporting the retained tissue specimen to a laboratory within the collection device 100. As described herein, various method embodiments of the present invention may also comprise filling the collection device 100 with a preservation fluid prior to operably engaging the proximal end cap 202 with the proximal end of the collection device 100 so as to preserve the retained tissue specimen within the collection device 100 when transporting the retained tissue specimen to the laboratory (such as a pathology laboratory).

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A tissue specimen collection system comprising:
a collection device adapted to be removably and serially engaged between a suction tube and a collection tube, the collection device comprising a distal end operably engaged with the collection tube and a proximal end operably engaged with the suction tube, the collection device defining a bore extending therethrough in coaxial relation with the suction tube and the collection tube, the bore being configured to allow fluid communication therebetween; and
a screen disposed within the bore of the collection device, the screen being configured to retain a tissue specimen drawn through the collection device by and towards the suction tube, the screen defining a plurality of apertures for allowing fluid communication between the collection tube and the suction tube, such that any fluid accompanying the tissue specimen is separated, and drawn through the screen and into the suction tube, while the tissue specimen is retained by the screen within the collection device.

2. The tissue specimen collection system according to claim 1, wherein the collection device is configured to be separable into a first segment including the distal end and a second segment including the proximal end and the screen, such that the retained tissue specimen is accessible for retrieval when the first segment is separated from the second segment.

3. The tissue specimen collection system according to claim 1, further comprising a distal end cap configured to sealingly engage the distal end of the collection device in a substantially fluid-tight manner such that the tissue specimen is retained within the collection device between the screen and the distal end cap when the collection device is removed from serial engagement between the suction tube and the collection tube.

4. The tissue specimen collection system according to claim 3, further comprising a proximal end cap configured to sealingly engage the proximal end of the collection device in a substantially fluid-tight manner such that a preservation fluid may be retained within the collection device between the distal end cap and the proximal end cap to preserve the tissue specimen when the collection device is removed from serial engagement between the suction tube and the collection tube.

5. The tissue specimen collection system according to claim 1, further comprising a preservation fluid reservoir configured to operably engage at least one of the proximal end and the distal end of the collection device when the collection device is removed from serial engagement between the suction tube and the collection tube; the preservation fluid reservoir defining an aperture for receiving at least one of the proximal end and the distal end of the collection device, the preservation fluid reservoir comprising a penetrable membrane configured to substantially seal the aperture so as to contain a preservation fluid therein, the membrane configured to be capable of being penetrated by at least one of the proximal end and the distal end of the collection device such that the preservation fluid is released into the bore defined in the collection device to preserve the tissue specimen retained therein.

6. The tissue specimen collection system according to claim 1, further comprising a manifold device configured to removably and serially engage the collection device between the suction tube and the collection tube.

7. The tissue specimen device according to claim 6, wherein the manifold device comprises at least one valve device operably engaged between the distal end of the collection device and the suction tube and configured to selectively allow fluid communication between the collection tube and the suction tube via the collection device.

8. The tissue specimen device according to claim 6, wherein the manifold device defines a reservoir in fluid communication between the proximal end of the collection device and the suction tube, the reservoir being configured to receive any fluid accompanying the tissue specimen such that the fluid may be drawn into the suction tube.

9. The tissue specimen device according to claim 6, wherein the manifold device comprises at least one substantially resilient structure configured to removably and serially engage the collection device between the suction tube and the collection tube.

10. The tissue specimen device according to claim 9, wherein the at least one substantially resilient structure is selected from the group consisting of:
a substantially-resilient polymer block defining an aperture being configured to receive at least one of the proximal and distal ends of the collection device;
a substantially-resilient C-clip being configured to receive at least one of the proximal and distal ends of the collection device; and
combinations thereof.

11. The tissue specimen device according to claim 8, further comprising a cartridge device configured to removably and serially engage the collection device between the reservoir and the collection tube, the cartridge device being slidably disposed in a cartridge chamber defined in a surface of the manifold device such that the cartridge device is selectively movable between a first position and a second position relative to the manifold device;
wherein the distal end of the collection device in direct fluid communication with the collection tube when the cartridge device is in the first position; and
wherein the collection device is accessible for removal from the cartridge device when the cartridge device is in the second position.

12. The tissue specimen collection system according to claim 7, wherein the manifold device is configured to removably and serially engage a plurality of collection devices in parallel relation between the suction tube and the collection tube and wherein the at least one valve device is further configured to selectively allow fluid communication between the collection tube and the suction tube via at least one of the plurality of collection devices.

13. The tissue specimen collection system according to claim 7, wherein the manifold device defines a bypass bore extending therethrough in coaxial relation with the suction tube and the collection tube, the bypass bore being configured to allow fluid communication therebetween, and wherein the at least one valve device is further configured to selectively allow fluid communication between the collection tube and the suction tube via the bypass bore so as to bypass the collection device.

14. The tissue specimen collection system according to claim 1, wherein the collection device comprises at least one indicia corresponding to an anatomical region from which the tissue specimen is drawn such that a user of the tissue specimen collection system may identify the anatomical region from which the tissue specimen was drawn during a medical procedure.

15. The tissue specimen collection system according to claim 14, wherein the at least one indicia is selected from the group consisting of:
an alphanumeric indicia;
a color;
a bar code;
a radio-frequency identification (RFID) device; and
combinations thereof.

16. The tissue specimen collection system according to claim 1, further comprising an organizer device configured to removably and serially engage the collection device between the suction tube and the collection tube, the organizer device defining a plurality of apertures therein for receiving the collection device when the collection device retaining the tissue specimen is removed from serial engagement between the suction tube and the collection tube, the plurality of apertures including one or more anatomical indicia corresponding thereto for indicating an anatomical region from which the tissue specimen is drawn such that the anatomical region is identifiable based at least in part on the anatomical indicia.

17. A method for collecting a tissue specimen, the method comprising:
providing a collection device adapted to be removably and serially engaged between a suction tube and a collection tube, the collection device defining a bore extending therethrough in coaxial relation with the suction tube and the collection tube, the bore being configured to allow fluid communication therebetween, the collection device comprising a screen disposed within the bore, the screen defining a plurality of apertures for allowing fluid communication between the collection tube and the suction tube;
operably engaging a distal end of the collection device with the collection tube;
operably engaging a proximal end of the collection device with the suction tube;
drawing a tissue specimen through the collection tube by and towards the suction tube; and
retaining the tissue specimen on the screen such that such that any fluid accompanying the tissue specimen is separated, and drawn through the screen and into the suction tube.

18. The method according to claim 17, further comprising:
separating the collection device into a first segment including the distal end and a second segment including the proximal end and the screen;
retrieving the retained tissue specimen from the screen; and
submerging the retrieved tissue specimen in a preservation fluid.

19. The method according to claim 17, further comprising operably engaging a distal end cap with the distal end of the collection device in a substantially fluid-tight manner such that the tissue specimen is retained within the collection device between the screen and the distal end cap when the collection device is removed from serial engagement between the suction tube and the collection tube.

20. The method according to claim 19, further comprising:
operably engaging a proximal end cap with the proximal end of the collection device in a substantially fluid-tight manner such that the tissue specimen is retained within the collection device between the distal end cap and the proximal end cap when the collection device is removed from serial engagement between the suction tube and the collection tube; and
transporting the retained tissue specimen to a laboratory within the collection device.
